(19) United States
(12) Patent Application Publication
(10) Pub. No.: US 2023/0146898 A1
(43) Pub. Date: May 11, 2023

(54) CIRCULATORY ASSIST DEVICE, CIRCULATORY ASSIST SYSTEM, AND RELATED METHODS

(71) Applicant: Second Heart Assist, Inc., Salt Lake City, UT (US)

(72) Inventor: Howard J. Leonhardt, Mission Viejo, CA (US)

(21) Appl. No.: 18/050,233

(22) Filed: Oct. 27, 2022

Related U.S. Application Data
(63) Continuation-in-part of application No. 17/698,287, filed on Mar. 18, 2022.
(60) Provisional application No. 63/263,133, filed on Oct. 27, 2021.

(51) Int. Cl. A61M 60/13; A61M 60/148

(52) U.S. Cl. CPC A61M 60/13; A61M 60/148; A61M 60/414

(57) ABSTRACT

A minimally invasive circulatory support device, system, and related methods. The circulatory assist devices, systems, and methods use low profile catheter-based techniques and provide temporary and chronic circulatory support depending on the needs of the patient. The circulatory assist device, systems, and methods include a stent cage and an impeller. The stent cage is formed of a first material that is sufficiently rigid to expand radially outward and press against an artery wall is sufficiently deformable to collapse within the outer sheath. The impeller includes at least one blade formed of a second material that is sufficiently rigid to expand and retain shape while rotating and assisting blood to flow within the artery and is sufficiently deformable to collapse within the outer sheath with the stent cage.

### CROSS-REFERENCE TO RELATED APPLICATION

This application claims the benefit under 35 U.S.C. § 119(e) of U.S. Provisional Patent Application Ser. No. 63/263,133, entitled “CIRCULATORY ASSIST DEVICE, CIRCULATORY ASSIST SYSTEM, AND RELATED METHODS,” filed Oct. 27, 2021; this application is also a continuation-in-part application of U.S. patent application Ser. No. 17/698,287, entitled “Circulatory Assist Pump,” filed Mar. 18, 2022, the contents of the entirety of each of which is hereby incorporated herein in its entirety by this reference.

### TECHNICAL FIELD

The application relates generally to medical devices, and more particularly to a system, apparatus, and associated methods for assisting a subject's heart to pump blood (e.g., a circulatory assist pump).

### BACKGROUND

Circulatory assist pumps and other devices may be used to assist a subject's heart to pump blood in order to address conditions such as heart disease.

U.S. 2021/0077687 A1 to Leonhardt (Published Mar. 18, 2021), the contents of which are incorporated herein by this reference, relates to a circulatory support platform utilizing an aortic stent pump, comprising a stent cage enabling open flow and an impeller within the stent cage. The circulatory support platform may facilitate blood circulation and pulsatility. The circulatory support platform may include shape memory materials to adjust the shape and size of the impeller blades. Additionally, the circulatory support platform may be wirelessly operated.

U.S. Pat. No. 8,617,239 to Reitan (Dec. 31, 2013), the contents of which are incorporated herein by this reference, relates to a catheter pump to be positioned in the ascending aorta near the aortic valve of a human being, comprising an elongated sleeve with a drive cable extending through the sleeve and connectable at its proximal end to an external drive source and a drive rotor near the distal end of the drive cable mounted on a drive shaft being connected with the drive cable. The drive rotor consists of a propeller enclosed in a cage and the propeller and the cage are foldable from an insertion position close to the drive shaft to an expanded working position, which are characterized by means for anchoring the drive rotor in the ascending aorta near the aortic valve after insertion. Also described is a method to position the pump of a catheter pump in the ascending aorta just above the aortic valve.

While the devices and systems of U.S. 2021/0077687 A1 to Leonhardt and U.S. Pat. No. 8,617,239 to Reitan operate well in many circumstances, there is still room for improvement.

The above-described background relating to circulatory assist pumps is merely intended to provide a contextual overview and is not intended to be exhaustive. Other contextual information may become apparent to those of ordinary skill in the art upon review of the following description, which includes example embodiments.

### BRIEF SUMMARY

Embodiments of the disclosure include a circulatory assist device, a circulatory assist system, and related methods.

In one embodiment, the disclosure provides a circulatory assist device including a stent cage and an impeller. The stent cage is formed of a first material that is sufficiently rigid to expand radially outward and press against an artery wall of an artery and that is sufficiently deformable to collapse within the outer sheath. The impeller includes at least one blade formed of a second material that is sufficiently rigid to expand and retain shape while rotating and assisting blood to flow within the artery and is sufficiently deformable to collapse within the outer sheath with the stent cage.

In another embodiment, the disclosure provides a circulatory assist system including a placement catheter and a circulatory assist device. The placement catheter includes an outer sheath. The circulatory assist device includes a stent cage and an impeller. The stent cage is formed of a first material that is sufficiently rigid to expand radially outward and press against an artery wall of an artery and that is sufficiently deformable to collapse within the outer sheath. The impeller includes at least one blade formed of a second material that is sufficiently rigid to expand and retain shape while rotating and assisting blood to flow within the artery and is sufficiently deformable to collapse within the outer sheath with the stent cage.

In a further embodiment, the disclosure provides a method of treating a subject in need thereof. The method includes making an incision in the subject to form an insertion point at an artery of the subject. The method also includes inserting a circulatory assist device into the artery of the subject while the circulatory assist device at least partially positioned within an outer sheath of a placement catheter. The circulatory assist device includes a stent cage formed of a first material that is sufficiently rigid to expand radially outward and press against an artery wall of an artery and that is sufficiently deformable to collapse within the outer sheath, and an impeller including at least one blade formed of a second material that is sufficiently rigid to expand and retain shape while rotating and assisting blood to flow within the artery and is sufficiently deformable to collapse within the outer sheath with the stent cage. The method further includes withdrawing the outer sheath from covering the stent cage and the impeller of the circulatory assist device. The method yet further includes after the circulatory assist device transitions into an expanded state with the stent cage expanded out against a wall of the artery and the blade expanding to an operational state, causing the impeller to rotate and assist the blood flow in the artery.

### BRIEF DESCRIPTION OF THE DRAWINGS

The disclosure is illustrated and described herein with reference to the various drawings, in which like reference numbers are used to denote like system components/method steps, as appropriate, and in which:

FIG. 1 illustrates an embodiment of a circulatory assist system in accordance with this disclosure;

FIG. 2 illustrates a perspective view of an embodiment of the circulatory assist catheter assembly of FIG. 1;

FIG. 3 illustrates a partial cross-sectional view of an embodiment of a circulatory assist device of FIGS. 1 and 2 in accordance with this disclosure;

FIGS. 4A-4C illustrate cross-sectional side and front views of the circulatory assist device of FIG. 3 in a closed configuration, an open configuration, and a partially open configuration, respectively;

FIGS. 5A-5C illustrate cross-sectional side views of embodiments of the impeller of FIG. 3;

FIG. 6 illustrates a cross-sectional side view of an embodiment of the impeller of FIG. 3;

FIGS. 7A-7D illustrate front views of various embodiments of the impeller of FIG. 3;

FIG. 8 illustrates an embodiment of the circulatory assist device including a wireless circulatory assist pump in an expanded state (open configuration), according to embodiments of this disclosure;

FIG. 9 illustrates the circulatory assist device of FIG. 8 with the wireless circulatory assist pump in a collapsed state (closed configuration);

FIG. 10 illustrates an embodiment of a control system;

FIG. 11 illustrates an embodiment of the circulatory assist device of FIGS. 8 and 9 including at least one vibrating component, according to embodiments of this disclosure; FIG. 11 illustrates the circulatory assist device of FIG. 10 positioned within an artery of a subject;

FIG. 12 illustrates another embodiment of a circulatory assist system including the circulatory assist device of FIG. 10 and a lower catheter inserted in the artery of the subject adjacent to the circulatory assist device;

FIG. 13 is a flowchart of a method for improving blood flow within an artery of a subject; and

FIG. 14 is a flowchart of a method for treatment.

### DETAILED DESCRIPTION

In various embodiments, this disclosure relates to devices, systems, and methods for providing temporary and chronic circulatory support for subjects (e.g., a mammal, such as a human) utilizing an impeller including at least one blade formed of a material configured to change a shape based on one or more conditions and configured to rotate within an artery (such as the aorta) of the subject to promote blood flow therein. Embodiments of the disclosure may be utilized in combination with or integrated into other circulatory assist systems. For example, the circulatory assist device of the disclosure may be utilized with or integrated into the circulatory assist pump and system described in U.S. 2021/0077687 A1 to Leonhardt and/or U.S. 2021/0008263 A1 to Leonhardt.

While, the description herein primarily discusses one-way shape memory materials, embodiments of the disclosure also include two-way shape memory materials. Additionally, one of ordinary skill in the art would understand and appreciate that there may be minor differences in the description below if a two-way shape memory material were utilized in embodiments of the disclosure described herein.

The circulatory assist device, system, and methods described herein involve a minimally invasive circulatory support platform that utilizes an aortic stent pump. For example, the circulatory support platform preferably uses a low profile, catheter-based technique. In operation, the circulatory support devices, systems, and methods described herein may require making an incision in the patient (e.g., in the groin region) at an insertion point for use thereof. As used herein, the term “insertion point” refers to an opening and/or blood vessel through which the circulatory assist device and/or system can be inserted into a subject (e.g., a mammal, such as a human). The opening may include an incision in the subject. Non-limiting examples of the insertion point may include: an incision in the subject's groin region, the subject's femoral artery, and the subject's femoral artery via an incision in the subject's groin region.

In certain embodiments, the circulatory device is configured to change between a closed configuration and an open configuration. The circulatory device remains in the closed configuration while the circulatory device is introduced into an artery, such as the femoral artery of the subject. The circulatory assist device is fed through a blood vessel in the subject to a desired location. For example, the circulatory assist device is fed through an incision in the groin-area of the subject, into the subject's femoral artery, and into a location just above the renal arteries within the subject's aorta (e.g., the descending aorta above the subject's kidneys). In embodiments, the circulatory assist device is configured to transition to open (e.g., deployed) configuration once the circulatory assist device is in the desired location, such as the subject's aorta. Once the circulatory assist device is open, the impeller of the circulatory assist device is activated to facilitate blood circulation through the subject's blood vessel(s) (e.g., descending aorta) and/or organs (e.g., heart). In embodiments, the circulatory assist device includes a stent cage that is deployed within the artery and configured to brace the artery wall and is configured to prevent the impeller from contacting the tunica intima while the impeller rotates within artery and the stent cage to facilitate the blood circulation.

As will be described in greater detail below, in some embodiments, the circulatory assist device incudes one or more impellers and/or impeller blades including shape memory material. In these embodiments, the memory material impeller blades are configured to selectively change to a predetermined shape (e.g., configuration), in one or more various ways, after being inserted within the subject's blood vessel(s) (e.g., descending aorta). For example, in some embodiments, the impeller blades made of shape memory material are configured to change shape, an amount of coil (such as via twisting), curvature, length, and/or overall form. In addition, in some embodiments, the shape memory material impeller blades and/or the impeller are configured to selectively change between a closed (e.g. undeployed) configuration and an open (e.g., deployed) configuration. Further, in some of these embodiments, the impeller blades made of shape memory material are configured to collapse within a casing (e.g., sheath introducer) while in the closed configuration and then expand to a predetermined configuration in the open configuration outside of the casing in response to the shape memory material reaching a predetermined temperature. The predetermined temperature being a temperature where the shape memory material transitions from a first state to a second state. In embodiments, the predetermined temperature is chosen from at, about, and/or above the transition temperature. For example, the transition temperature for shape memory alloys is the austenite transition temperature (TA), and the transition temperature for shape memory polymers may be either the high glass transition temperature (TG) or the intermediate melting temperature (TM). Temporary stresses and/or strain within the shape memory material impeller blades and/or impeller may be removed in response to the shape memory material being at, about, and/or above the transition temperature. In addition, superelasticity (e.g., for shape memory alloys) and visco-elasticity (e.g., for shape memory polymers) of shape memory material impellers and/or impeller blades may facilitate constructions of circulatory assist devices and systems with fewer moving parts.

The blades and/or the impeller may be made of a shape memory material that has a transition temperature (e.g., TA for shape memory alloys, or either TG or TM for shape memory polymers) tailored to be at, about, or slightly below the internal body temperature of the subject (e.g., a mammal, such as a human). For example, the transition temperature of the shape memory material may be from about 35° C. to about 40° C. As described in U.S. Pat. No. 4,283,233 to Goldstein et al. (Aug. 11, 1981), the contents of which is incorporated herein by this reference, shape memory alloys, such as Nitinol, can be engineered to include an austenite transition temperature just below the normal human body temperature, thus allowing for the shape memory effect and super elasticity effects to occur in conjunction with the circulatory assist devices and systems of this disclosure due to body heat. Similarly, U.S. Patent Pub. No. 2009/0248141 to Shandas et al. (Published Oct. 1, 2009), incorporated herein by this reference, describes a method of tailoring the transition temperature of shape memory polymers to allow recovery at, above, or below the human body temperature of 37° C.

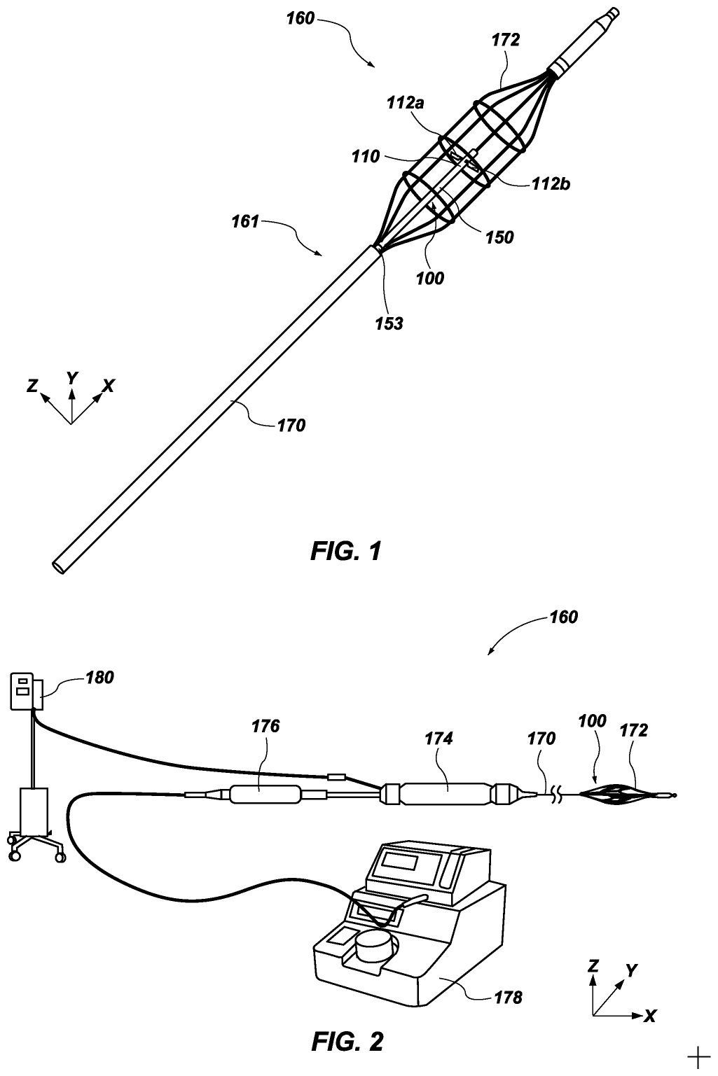

FIG. 1 illustrates an embodiment of a circulatory assist system 160 in accordance with this disclosure.

Referring to FIG. 1, in embodiments, the circulatory assist system 160 includes a circulatory assist catheter assembly 161, at least one sheath driveline 174, 176, a motor drive control unit 178, and a power supply 180. As will be described in greater detail below, in embodiments, the circulatory assist catheter assembly 161 includes a placement catheter 170, the stent cage 172, and a circulatory assist device 100. In embodiments, the circulatory assist catheter assembly connects to the at least one sheath driveline 174, 176 via the placement catheter 170, with the stent cage 172 and the circulatory assist device 100 distal to the connection to the at least one sheath driveline 174, 176, such as proximate/adjacent to a distal end of the circulatory assist catheter assembly 161. The at least one sheath driveline includes a sheath driveline 174 configured to rotate the impeller 110 (refer to FIGS. 2-7D) of the circulatory assist device 100 and/or control relative movement between the casing 150 (refer to FIG. 2) and the placement catheter 170, such as by controlling axial movement relative to the casing 150 of at least one component chosen from the casing 150 and the placement catheter 170, and in particular, an outer sheath 153 of the placement catheter 170. In some embodiments, the at least one sheath driveline includes a second sheath driveline 176 configured to facilitate movement and/or rotation of the casing 150 (refer to FIG. 2) and/or the placement catheter 170. In embodiments, the motor drive control unit 178 is configured to facilitate movement and/or rotation of the impeller 110 (refer to FIG. 2), the casing 150 (refer to FIG. 2), and/or the placement catheter 170, such as via the at least one sheath driveline 174, 176. The power supply 180 (e.g., medical grade UPS) is configured to facilitate transport and provide power to the circulatory assist system 160.

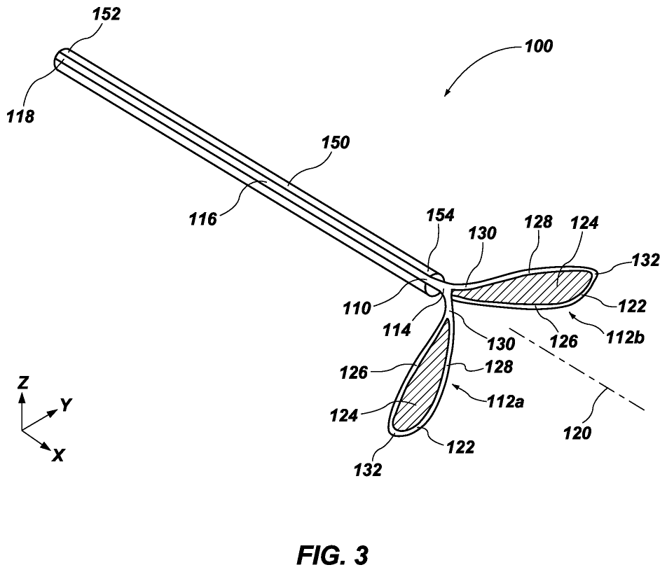

FIG. 2 illustrates a perspective view of an embodiment of the circulatory assist catheter assembly 161 of FIG. 1. Referring to FIGS. 1 and 2, in embodiments, the impeller 110 (FIG. 1) of the circulatory assist device 100 of the circulatory assist catheter assembly 161 is configured to be operated (e.g., rotated) mechanically via a shaft 116 (refer to FIG. 3) that is operably coupled to the motor drive control unit 178. In additional embodiments, the impeller 110 is configured to operate (e.g., rotated) wirelessly.

In embodiments, any of the circulatory assist device 100 including the impeller 110, and the stent cage 172 to selectively change to a desired predetermined “remembered” shape after being positioned in a desired location within the subject's artery/blood vessel(s). In addition, in embodiments, as described herein, the impeller 110 is configured for selectively change the length, shape, an amount of coil (such as via twisting), curvature and/or overall form thereof. In addition, superelasticity (e.g., for shape memory alloys) and visco-elasticity (e.g., for shape memory polymers) of shape memory material impellers may facilitate constructions of circulatory assist devices and systems with fewer moving parts.

In embodiments, the stent cage 172 is configured to transition between an open (e.g., deployed) state and a closed (e.g., stowed or collapsed) state. The stent cage 172 is configured to be introduced into the subject while in the closed state, along with the circulatory assist device 100 via the placement catheter 170. In response to the stent cage 172 being positioned in a desired location within the patient (e.g., just above the renal arteries within the subject's aorta), the stent cage 172 is configured to expand to the open state. The stent cage 172 is configured to facilitate operation of the circulatory assist device 100 and to guard the surrounding tissue from being impacted by the impeller 110, which may reduce the chance that the impeller 110 damages the surrounding tissue. In embodiments, the impeller 110, while in an open, deployed state is positioned within the stent cage 172 that shields, e.g., the subject's aortic tissue from the impeller 110. In other embodiments, other guards, shields, or cages are utilized to protect the subject's tissue from the impeller 110. In embodiments, the stent cage 172 is configured for a highly open flow. The stent cage 172 is sized and made of a material that provides for stability against the artery wall of the subject, such as the aortic wall. In embodiments, the stent cage 172 is configured to exert sufficient pressure on the artery wall to secure the stent cage 172 and the impeller 110 in a fixed position within the artery. For example, in some embodiments, the stent cage 172 exerts sufficient right radial force to distend an artery, such as the aorta, two (2) mm, facilitating extra flow and providing a safety area that stabilizes a position of circulatory assist device 100. In embodiments, the stent cage 172 is adapted to be sufficiently rigid to maintain a secure open (e.g., deployed) position braced against the subject's artery, sufficiently flexible to enable fluctuations due to natural pulsatility of the subject's blood vessel(s).

Maintaining vessel wall motion during natural pulsatility may facilitate aortic protein expressions such as Klotho that promote multiple organ health especially kidney health and avoid plaque formation. Maintaining vessel wall motion during natural pulsatility may also improve blood pressure and hemodynamics. The benefits of natural pulsatility are discussed in the following article, the contents of which are incorporated herein by this reference: Why pulsatility still matters: a review of current knowledge, Davor Barić, Croatian Medical Journal, Volume 55(6), December 2014, pages 609-620, DOI: 10.3325/cmj .2014.55.609.

FIG. 3 illustrates a partial cross-sectional view of an embodiment of the circulatory assist device 100 of FIGS. 1 and 2 in accordance with this disclosure. Referring to FIG. 3, in embodiments, the circulatory assist device 100 includes an impeller 110 and a casing 150 (e.g., an introducer sheath). The casing 150 includes a tubular shape and is configured to receive at least a portion of the impeller 110.

The impeller 110 is typically configured to selectively change shapes in one or more various ways. For example, the impeller 110 may be configured to selectively change to a predetermined open (e.g., deployed) configuration, illustrated in FIG. 2.

In embodiments, the impeller includes a shaft 116 and at least one blade 112a, 112b. The shaft 116 includes a proximal end 118 configured to be proximal to an assertion point of the circulatory assist device 100 and coupled to the at least one sheath driveline to be rotated thereby. The shaft 116 is at least partially positioned within the casing 150 and is configured to rotate relative to the casing 150 and to cause the at least one blade 112a, 112b to rotate.

In the embodiment illustrated, the at least one blade 112a, 112b includes two blades 112a, 112b (collectively, the “blades 112”). The at least one blade 112a, 112b extend from a distal end 114 of the shaft 116, which is configured to be distal to the insertion point and distal to the at least one sheath driveline. Although illustrated as including two blades 112, the at least one blade 112a, 112b may include any number of blades 112 (e.g., one, two, three, or more). The impeller 110 includes a central longitudinal axis 120, defined by the shaft 116, about which the shaft 116 and the blades 112 rotate.

In embodiments, such as the embodiment illustrated in FIG. 3, the blades 112 include an exterior portion 122 defining an exterior boundary of the blades 112 and an interior portion 124 positioned within and/or between the exterior portion 122. The blades 112 are configured to rotate relative to the casing 150 and the artery that the circulatory assist device 100 is positioned within. Accordingly, each of the blades 112 includes a rotationally leading edge 126 on a first side thereof and a rotationally trailing edge 128 opposite the rotationally leading edge 126 on a second side thereof. The blades 112 additionally include a proximal end 130 positioned near the shaft 116 of the impeller 110, and a distal end 132 opposite the proximal end 130 of the blades 112 and arranged distal to the shaft 116. In some embodiments, each of the blades 112 is connected to the distal end 114 of the shaft 116 at or adjacent to the proximal end 130 thereof. In embodiments, the blades 112 are formed with the shaft 116 or a portion thereof as a unitary structure. In other embodiments, the blades 112 are joined to the shaft 116.

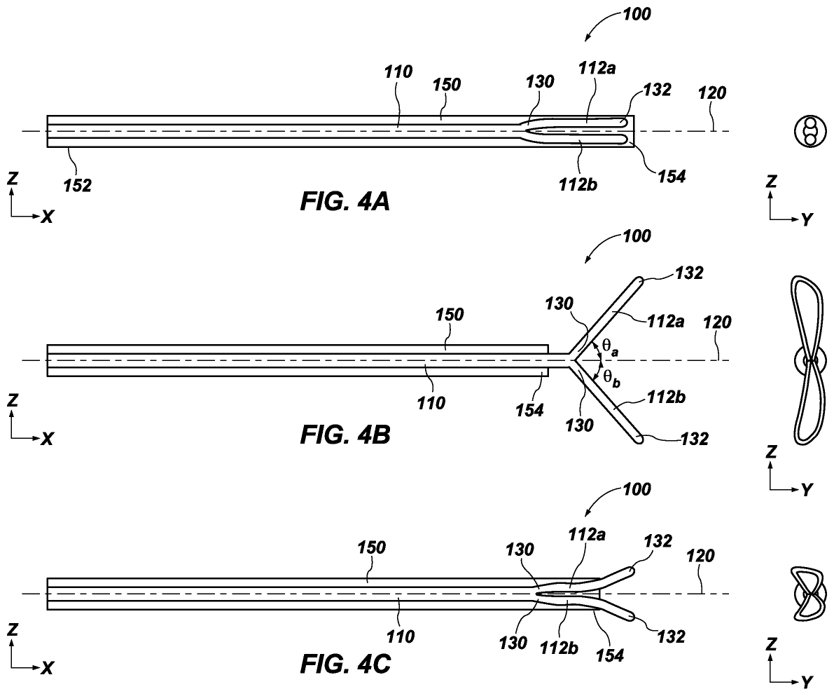

In embodiments, the impeller 110 is configured to transition between an open (e.g., deployed) configuration, such as the arrangement shown in FIG. 3, and a closed (e.g., stowed) configuration (see for example the arrangement of FIG. 4A). In embodiments, to transition between the open configuration and the closed configuration, the impeller 110 is configured to move relative to the casing 150 in an axial direction (e.g., along the central longitudinal axis 120). For example, in embodiments, in the open condition, the at least one blade 112a, 112b is adjacent to the casing 150 with the distal end 114 of the shaft 116 at least partially extends from the casing 150 and, in the closed condition, the at least one blade 112a, 112b and the distal end 114 of the shaft 116 are positioned within the casing 150, such as by being pulled and drawn into the casing 150.

FIGS. 4A-4C illustrate cross-sectional side and front views of the circulatory assist device 100 of FIG. 3 in a closed configuration, an open configuration, and a partially open configuration, respectively. In embodiments, the closed configuration (FIG. 4A) is arranged to facilitate introduction and removal of the circulatory assist device 100, and in particular, the impeller 110 into and from the subject's blood vessel(s)/artery (e.g., descending aorta). The open configuration (FIG. 4B) is arranged to facilitate blood flow through the subject's blood vessel(s)/artery (e.g., descending aorta), and in particular, is arranged to deploy the at least one blade 112a, 112b and position the at least one blade 112a, 112b for rotation about the central longitudinal axis 120 with the at least one blade 112a, 112b oriented and angled to cause blood flow within the artery. The partially open configuration (FIG. 4C) includes any orientation of the impeller 110, and in particular, the at least one blade 112a, 112b, between the closed configuration (FIG. 4A) and the open configuration (FIG. 4B).

Referring collectively to FIGS. 4A-4C, as noted above, in embodiments, the impeller 110 is configured to move in an axial direction (e.g., along the central longitudinal axis 120) relative to the casing 150. In some embodiments, axial movement between the impeller 110 and the casing 150 facilitates the impeller 110 transitioning between the closed configuration (FIG. 4A), the open configuration (FIG. 4B), and the partially opened configuration (FIG. 4C). For example, the distal end 114 of the shaft 116 and the at least one blade 112a, 112b may be extended from or retracted into the casing 150 and/or the casing 150 may be extended over or retracted from covering the distal end 114 of the shaft and the at least one blade 112a, 112b .

In embodiments, the impeller 110, and in particular, the blades 112 are formed of at least one shape memory material (e.g., shape memory polymer or shape memory alloy) that is configured to selectively change from a first predetermined shape to a second predetermined shape (e.g., an closed configuration) in various ways. For example, in embodiments, the blades 112 are configured to change shape, an amount of coil, curvature, length, and/or overall form.

In some embodiments, the impeller 110, and in particular, the blades 112, include temperature sensitive shape memory material configured to selectively change shape in response the temperature of the shape memory material reaching a predetermined temperature. In embodiments, the predetermined temperature is the transition temperature where the shape memory material changes from a first shape to a second shape. In some embodiments, the predetermined temperature is chosen from being at, about, or above the transition temperature (e.g., TA for shape memory alloys, and either TG or TM for shape memory polymers).

To set a predetermined desired “remembered” shape and/or configuration, the shape memory material of the impeller 110, and in particular, the blades 112, is heated to a setting temperature that is above (e.g., at least one degree Celsius and/or 5% or more above) the transition temperature of the shape memory material, and the blades 112 are then arranged/positioned (e.g., deformed, oriented) to the desired predetermined “remembered” shape and/or configuration, such as for the open condition. For example, the blades 112 may be arranged/positioned in the open configuration, as illustrated in FIG. 4B, while the temperature thereof is at or above the setting temperature. Once the blades 112 are arranged/positioned in the desired “remembered” configuration for the open condition, the blades 112 are cooled, while in the desired “remembered” configuration until the temperature is below the transition temperature to set the predetermined “remembered” configuration in the blades 112.

In embodiments, while the shape memory material of the impeller 110, and in particular, the blades 112 is below the transition temperature, the impeller 110, and in particular, the blades 112 are arranged/positioned (e.g., deformed, oriented) into any variety of “temporary” configurations and/or shapes. For example, in embodiments, the impeller 110, and in particular, the blades 112, are arranged/positioned into the closed withdrawn configuration, such as the configuration illustrated in FIG. 4A. In embodiments, once the configurations are set, in response to being at, about and/or above the transition temperature, the shape memory material of the impeller 110 changes from the closed configuration to the open configuration. In embodiments, the impeller 110 is constrained from opening to the open configuration by the casing 150, while the distal end 114 of the shaft 116 is within the casing 150 and the impeller 110 reaches the predetermined temperature. In response to relative movement between the impeller 110 and the casing 150 exposing the distal end 114 of the shaft 116 and the blades 112 from the casing 150 while the impeller 110 is still at or above the predetermined temperature, the impeller 110 expands to the open condition. As previously discussed, in embodiments, the predetermined temperature is the transition temperature (e.g., TA for shape memory alloys, and either TG or TM for shape memory polymers) of the shape memory material may be tailored to be about internal body temperature of the subject, such as from about 35° C. to about 40° C. (e.g., about 37° C.). In embodiments, the impeller 110 is configured to include a transition temperature relative to the internal body temperature with the setting temperature being sufficiently above the internal body temperature to prevent the shape of the open condition from being reset while the impeller 110 is positioned within a subject's body.

As noted above, in some embodiments, the shape memory material exhibits a two-way shape memory effect. For example, the shape memory material of the impeller 110 and/or the blades 112 includes two separate predetermined “remembered” shapes and/or configurations. The shape memory material of the impeller 110 and/or the blades 112 includes a first predetermined “remembered” shape and/or configuration above a transition temperature (e.g., TA for shape memory alloys, and either TG or TM for shape memory polymers) and a second predetermined “remembered” shape and/or configuration below the transition temperature. In some embodiments, the first predetermined “remembered” configuration (e.g., above the transition temperature) includes the impeller 110 and/or the blades 112 in the open configuration (FIG. 4B), and the second predetermined “remembered” configuration (e.g., below the transition temperature) includes the impeller 110 and/or the blades 112 in the closed configuration (FIG. 4A). Similar to the embodiments disclosed above, the transition temperature of two-way shape memory materials may be tailored to be about internal body temperature of the subject, such as from about 35° C. to about 40° C. (e.g., about 37° C.).

Referring now to FIG. 4A, the impeller 110 is depicted in the closed (e.g., stowed) configuration. In the embodiment illustrated, the blades 112 are in the “temporary” configuration when the impeller 110 is in the closed configuration. As previously discussed, the closed configuration may facilitate introduction and removal of the impeller 110 into the subject's blood vessel(s) (e.g., the subject's femoral artery and/or aorta).

While the impeller 110 is in the closed configuration, the impeller 110, including the blades 112, is at least partially within the casing 150. In some embodiments, the blades 112 (including the distal end 132) are positioned entirely within the casing 150 while the impeller 110 is in the closed configuration, as shown in FIG. 4A. Before inserting the circulatory assist device 100 within the subject, the impeller 110 is inserted, at least partially, into the proximal end 152 of the casing 150. For example, in embodiments, the blades 112 collapse inward together with the distal end 132 of the blades 112 and/or at least a portion of the blades 112 (e.g., a portion of or the entire blades 112) at least substantially aligned with the shaft 116 of the impeller 110 and while within the casing 150, and the impeller 110 is fed into the casing 150 until the distal end 132 of the blades 112 are near the distal end 154 of the casing 150. Additionally, the blades 112 and/or the impeller 110 are configured to selectively change to a predetermined open configuration (FIG. 4B) upon removal from the casing 150 and in response to reaching the predetermined temperature/remaining above the predetermined temperature. For example, when the impeller 110 is at or above the transition temperature (e.g., the subject's internal body temperature), the impeller 110, and in particular, the blades 112, is biased toward the open configuration due to the material structure of the “remembered configuration” established in the memory material. However, as illustrated in FIG. 4A, sidewalls of the casing 150 prevent opening (e.g., expansion) of the impeller 110 to the open configuration, and in particular, prevent the blades 112 from opening, even when the memory material reaches about or above the transition temperature, until the blades 112 are removed from the casing 150.

In response to the casing 150 and/or the impeller 110 being introduced into the subject's blood vessel(s) and fed through the subject's blood vessel(s) to a desired location, the impeller 110 reaching about or above the transition temperature, and the impeller 110 being moved relative to the casing 150 to be removed at least partially therefrom, the impeller 110 transitions from the closed configuration to the predetermined open (e.g., deployed) configuration, as illustrated in FIG. 4B. For example, the casing 150 and/or the impeller 110 is fed through an incision adjacent the subject's groin, through the subject's femoral artery, and into the subject's aorta. In response to the casing 150 and/or impeller 110 being positioned at or about just above the renal arteries, the impeller 110 is transitioned to an open configuration, such as by reaching about or above the transition temperature and being positioned axially offset from the casing 150 (i.e. the casing 150 is no longer axially aligned with and radially outward of the blades 112).

Referring now to FIG. 4B, the impeller 110 is depicted in an open condition. As noted above, in embodiments, relative axial movement between the impeller 110 and the casing 150 results in the blades 112 extending from the casing 150 and being exposed within an artery of the subject, which allows the blades 112 to expand into the open condition in response to reaching about or above the transition temperature. For example, the relative movement between the impeller 110 and the casing 150 is accomplished by at least one movement chosen from the impeller 110 being extended from the casing 150 and the casing 150 being retracted from the impeller 110. In the open configuration, the proximal end 130 of the blades 112 extend beyond the distal end 154 of the casing 150 with the entirety of the blades 112 external to the casing 150. Again, the blades 112 and/or the impeller 110 are configured to selectively change to the predetermined open configuration. For example, when the impeller 110 is at about or above the transition temperature (e.g., the subject's internal body temperature), the impeller 110, and in particular, the blades 112, is biased toward the open configuration due to the material structure of the “remembered configuration” established in the memory material. Because the blades 112 are external to the casing 150, the blades 112 are not constrained by the casing 150 and transition to the open configuration, as shown.

In embodiments, in the open configuration, each blade 112a, 112b of the impeller 110 is oriented at a respective angle (θa), (θb) measured from the central longitudinal axis 120 of the impeller 110. For example, in some embodiments, each angle (θa), (θb) may be less than or equal to 90° and/or forms an acute angle with the central longitudinal axis 120 while extending away from the distal end 154 of the casing 150, such as from about 5° to about 85°, from about 10° to about 80°, from about 15° to about 75°, from about 20° to about 70°, from about 25° to about 65°, from about 30° to about 60°, from about 35° to about 55°, from about 40° to about 50°, or about 45°. In some of these embodiments, one or more blades (e.g. 112a, 112b) form an obtuse angle with the casing 150. In some embodiments, the blades 112 are oriented at the same angle (θ) measured from the central longitudinal axis 120 of the impeller 110. For example, in some embodiments, the angles (θa), (θb) of the blades 112a, 112b are both oriented at about 60° measured from the central longitudinal axis 120 of the impeller 110. In additional embodiments, one or more blades (e.g., 112a and 112b) is oriented at a different angle measured from the central longitudinal axis 120 of the impeller 110 than other blades 112, with the angle (θa) of one blade 112a is different than the angle (θb) of another blade 112b. For example, in one embodiment, the angle (θa) of blade 112a is about 30°, and the angle (θb) of blade 112b is about 45°. For blades 112 that are generally non-planar (e.g., curved), the angle (θ) of the blade 112a is determined from a straight line defined from the proximal end 130 to the distal end 132 of the respective blade 112.

In embodiments, to transition the impeller 110 from the open configuration back into the closed configuration (e.g., for removal from the subject), relative movement between the impeller 110 and the casing 150 is caused in the axial direction to position the impeller 110, and in particular the blades 112, at least partially within the casing 150. For example, the relative movement between the impeller 110 and the casing 150 is accomplished by at least one movement chosen from the impeller 110 being retracted into the casing 150, such as the proximal end 130 of the blades 112 being moved relative to the distal end 154 of and into the casing 150 and the casing 150 being moved over the impeller 110. In embodiments, the blades 112 and/or the impeller 110 are retracted, at least partially, into the casing while the impeller 110 is stall at, about, or above the transition temperature (e.g., the subject's internal body temperature), biasing the blades 112 out of the open configuration and toward the closed configuration. In embodiments, the rigidity of the casing 150 is higher than that of the memory material. In some embodiments, a portion of the blades 112 that remains external to the casing 150 remains partially expanded, as shown in FIG. 4C. As the impeller 110 moves in the axial direction relative to the casing 150, the distal end 154 of the casing 150 provides an inward force against a rear surface of the blades 112 causing the blades 112 to deflect and collapse inward towards the central longitudinal axis 120. For example, the proximal end 130 of the blades 112 and/or at least a portion of the blades 112 (e.g., a portion of or the entire blades 112) are at least substantially aligned with the central longitudinal axis 120 of the impeller 110.

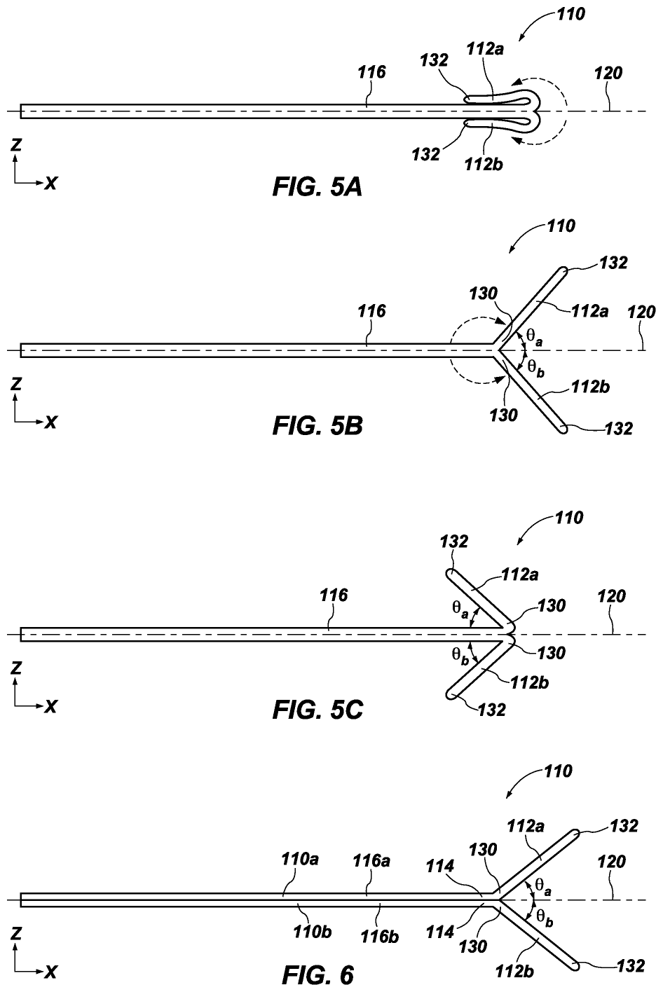

FIGS. 5A-5C illustrate cross-sectional side views of the impeller 110 of FIG. 3. Referring collectively to FIGS. 5A-5D the blades 112 may exhibit substantial flexibility to be deflected by the casing 150 and to be stored within the casing 150, while also exhibiting substantial rigidity to maintain the “remembered” configuration while in the open configuration and rotating to cause blood flow within an artery. As previously discussed, in embodiments, the blades 112 are configured to change shape, an amount of coil, curvature, length, and/or overall form. As previously discussed, in embodiments, the blades 112 exhibit substantial malleability to facilitate manipulation of the blades 112 while setting the predetermined “remembered” configuration (e.g., the open configuration (FIG. 4B), FIG. 5B, FIG. 5C). In addition, the blades 112 exhibiting substantial malleability to facilitate manipulating the “temporary” configuration of the blades 112 after the predetermined “remembered” configuration has been set. For example, in embodiments, the blades 112 being flexible and deformable facilitate transitioning between the closed configuration (FIG. 4A) and the open configuration (FIG. 4B), even if the shape memory material has already reached the transition temperature (e.g., when the impeller 110 is within the subject).

Referring to FIG. 5A, in embodiments, the blades 112 are sufficiently flexible/deformable for the distal ends 132 of the blades 112 to be bent and/or deformed back towards the shaft 116 of the impeller 110 with exterior surfaces of the blades 112 contacting the shaft 116. In other words, the distal ends 132 of the blades 112 of the impeller 110 are configured to be oriented up to 180° from the central longitudinal axis 120 of the impeller 110. In addition, the distal ends 132 and/or the blades 112 may be oriented in any direction (e.g., in the X-Z plane) for the predetermined “remembered” configuration. In some embodiments, the predetermined “remembered” configuration includes the blades 112 positioned with the distal ends 132 of the blades 112 oriented toward the shaft 116 of the impeller 110 (e.g., in the negative X-direction forming an acute angle with the shaft 116, FIG. 5C). In other embodiments, the predetermined “remembered” configuration includes the distal ends 132 of the blades 112 at least substantially aligned with the central longitudinal axis 120 of the impeller 110. In additional embodiments, the predetermined “remembered” configuration includes the blades 112 positioned with the distal ends 132 of the blades 112 oriented in the Z-direction or away from the shaft 116 of the impeller (e.g., in the positive X-direction forming an obtuse angle with the shaft 116, FIG. 5B). In further embodiments, the predetermined “remembered” configuration includes the blades 112 positioned away from the shaft 116 of the impeller 110 and at least substantially aligned with the central longitudinal axis 120 of the impeller 110. In still further embodiments, the blades 112 of the impeller 110 are arranged in any combination of the configurations outlined above.

FIG. 5B illustrates an embodiment of the impeller 110 and the blades 112 in a predetermined open configuration. In embodiments, if the blades 112 are bent backward such that the distal ends 132 of the blades 112 are oriented toward the shaft 116 of the impeller 110, as shown in FIG. 3A, and the temperature of the blades 112 and/or the impeller 110 is at about or above the transition temperature, the blades 112 will “spring” back to the predetermined open configuration shown in FIG. 3B (e.g. oriented in the positive X-direction with the blades forming an obtuse angle with the shaft 116). As previously discussed, each blade 112a, 112b of the impeller 110 may be oriented at the respective angles (θa), (θb) measured from the central longitudinal axis 120 of the impeller 110.

FIG. 3C illustrates another embodiment of the impeller 110 and the blades 112 in a predetermined open configuration. In the embodiment illustrated in FIG. 3C, in the predetermined open configuration, the distal ends 132 and/or the blades 112 are shown oriented toward the shaft 116 of the impeller 110 (e.g., oriented in the negative X-direction with the blades 112 forming an acute angle with the shaft 116). As previously discussed, each blade 112a, 112b of the impeller 110 may be oriented at the respective angles (θa), (θb) measured from the central longitudinal axis 120 of the impeller 110.

The impeller 110 may include one or more materials and/or structures and may be made in a variety of ways.

The impeller 110 may include any suitable shape memory material, such as a biocompatible shape memory alloy or biocompatible shape memory polymer. For example, the impeller may include a shape memory alloy such as Nitinol, and/or one or more shape memory polymers such as polyether, polyacrylate, polyamide, polysiloxane, polyurethane, polyethylene, methyl- methacrylate (MMA), polyethylene glycol (PEG), polyethylene glycol dimethacrylate (PEGDMA), polyether amide, polyether ester, or urethane-butadiene copolymer, or any combination thereof In addition, the impeller 110 may include one or more additional materials such as poly-paraphenylene terephthalamide (“para-aramid,” KEVLAR® or TWARON®) or similar material.

In some embodiments, the impeller 110 includes one or more materials and/or structures. The impeller 110 may comprise a unitary (e.g., monolithic) structure made of shape memory material that is formed as a single, unitary structure. For example, the impeller 110 may be formed from a single shape memory material (e.g., Nitinol) wire. In some embodiments, an end of the single shape memory material wire is separated (e.g., machined) to form the blades 112 and the remaining (e.g., un-machined) portion of the single shape memory material wire forms the shaft 116. The shape memory material (e.g., Nitinol) wire may be about 4 American Wire Gauge (AWG) and smaller. For example, the shape memory material wire may be from about 4 AWG (5.189 mm diameter) to about 30 AWG (0.255 mm diameter), such as from about 10 AWG (2.588 mm diameter) to about 20 AWG (0.812 mm diameter), and more particularly from about 14 AWG (1.628 mm diameter) to about 18 AWG (1.024 mm diameter), such as about 16 AWG (1.291 mm diameter).

FIG. 6 illustrates a cross-sectional side view of an embodiment of the impeller 110 of FIG. 3. Referring now to FIG. 6, in embodiments, the impeller 110 includes wo or more materials and/or structures combined to form the impeller 110. For example, the impeller 110 may include two or more shape memory material (e.g., Nitinol) wires. In some embodiments, such as the embodiment illustrated in FIG. 6, the impeller 110 includes a first impeller structure 110a and a second impeller structure 110b. The first and second impeller structures 110a, 110b include respective shafts 116a, 116b and respective blades 112a, 112b. The first impeller structure 110a includes the blade 112a and the shaft 116a, the blade 112a and the shaft 116a being a single unitarily formed structure. The second impeller structure 110b includes the blade 112b and the shaft 116b, the blade 112b and the shaft 116b being a single unitarily formed structure. The first impeller structure 110a may be joined to the second impeller structure 110b to form the impeller 110, such as by a bonding process (e.g. welding). The first impeller structure 110a and/or the second impeller structure 110b each include a shape memory material (e.g., Nitinol) wire. The shape memory material wire may be about 8 American Wire Gauge (AWG) and smaller. For example, the shape memory material wire may be from about 8 AWG (3.264 mm diameter) to about 36 AWG (0.127 mm diameter), such as from about 16 AWG (1.291 mm diameter) to about 26 AWG (0.405 mm diameter), and more particularly from about 18 AWG (1.024 mm diameter) to about 24 AWG (0.511 mm diameter), such as about 22 AWG (0.644 mm diameter).

In additional embodiments, the blades 112 of the impeller 110 are formed separately from and joined to the shaft 116 of the impeller 110. For example, in some embodiments, the blades 112 are formed from one or more shape memory materials, and the shaft 116 are formed from one or more additional shape memory materials.

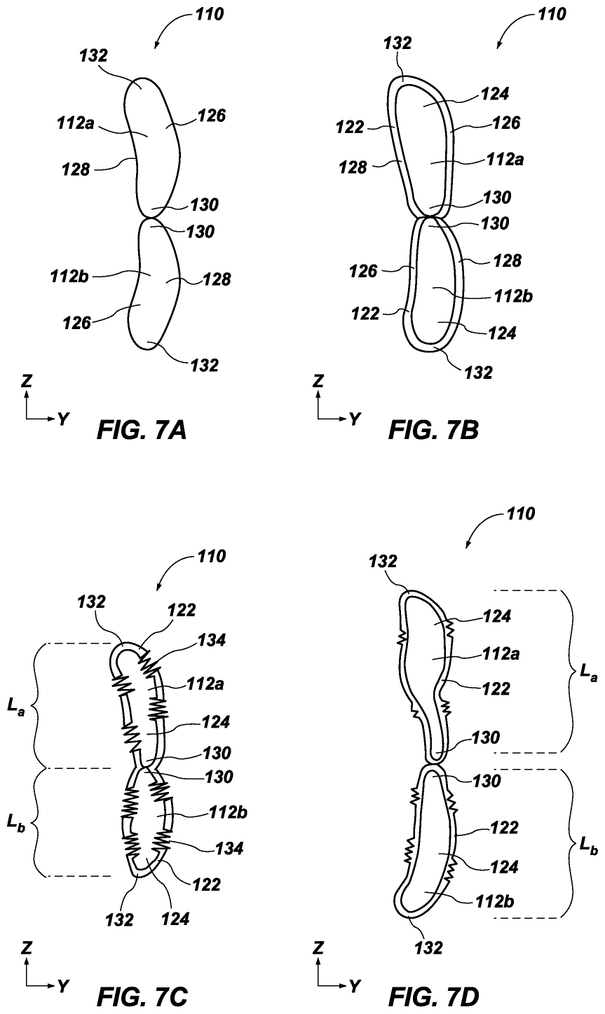

FIGS. 7A-7D illustrate front views of various embodiments of the impeller 110 of FIG. 1. Referring collectively to FIGS. 7A-7D, in embodiments, each of the blades 112 includes a substantially elliptical shape, such as a dragonfly-wing shape. In some embodiments, the blades 112 an airfoil shape. For example, in some embodiments, the rotationally/circumferentially leading edge 126 is more rounded and thicker than the rotationally/circumferentially trailing edge 128. In some of these embodiments, each of the blades 112 includes a tear drop shaped cross-section. Additionally, the blades 112 may be substantially planar, substantially curved, or any other shape suitable shape for promoting blood flow through the subject's artery in response to rotation of the impeller 110, while reducing hemolysis and heat. In some embodiments, the impeller 110 is rotated at or below a low revolutions per minute (RPM), such as from 1,500 RPM to 9,000 RPM. In some embodiments, the impeller 110 is rotated from 1,500 RPM to 4,500 RPM.

Referring now to FIG. 7A, in some embodiments, each of the blades 112 are formed from either a single shape memory alloy or a single shape memory polymer. In some embodiments, the blades 112 are formed of a shape memory alloy or polymer that is the same as the material of the respective shaft 116, and in other embodiments, the material of each of the blades 112 is different than the material of the respective shaft 116 of the impeller 110. In one or more embodiments, the impeller 110 includes a single shape memory material (e.g., Nitinol) wire that is machined and/or formed (e.g., flattened) on the distal end 114 (refer to FIG. 3) of the impeller 110 to form the blades 112.

FIG. 7B illustrates an embodiment with blades 112 formed from multiple materials. Referring now to FIG. 7B, in embodiments, each of the blades 112 includes an exterior portion 122 and an interior portion 124. In embodiments, the exterior portion 122 of the blades 112 includes one material and/or structure, and the interior portion 124 of the blades includes a separate material and/or structure that is positioned within and joined to the exterior portion 122. In some embodiments, the exterior portion 122 of the blades 112 defines a blade frame formed of a first material, and the interior portion 124 defines a blade body that fits within the frame formed of a second material connected to and interposed between the blade frame. In some of these embodiments, the second material is the same material as the first material. For example, in some embodiments, the exterior portion 122 and the interior portion 124 are each formed of a shape memory alloy (e.g., Nitinol) or a shape memory polymer (e.g., polyether, polyacrylate, polyamide, polysiloxane, polyurethane, polyethylene, methyl- methacrylate (MMA), polyethylene glycol (PEG), polyethylene glycol dimethacrylate (PEGDMA), polyether amide, polyether ester, or urethane-butadiene copolymer).

In additional embodiments, the second material is formed from a different material than the first shape memory material. For example, in some embodiments, the exterior portion 122 is formed of a shape memory alloy (e.g., Nitinol) or and the interior portion 124 is formed of a shape memory polymer (e.g., polyether, polyacrylate, polyamide, polysiloxane, polyurethane, polyethylene, methyl- methacrylate (MMA), polyethylene glycol (PEG), polyethylene glycol dimethacrylate (PEGDMA), polyether amide, polyether ester, or urethane-butadiene copolymer) and/or one or more electroactive materials such as poly-paraphenylene terephthalamide (KEVLAR® or TWARON®) or similar material.

Referring collectively to FIGS. 7C-7D, as noted above, in some embodiments, the blades 112 are configured to selectively change length, shape, an amount of coil, curvature and/or overall form. In some embodiments, the exterior portion 122 includes a temperature sensitive shape memory alloy and the interior portion 124 includes one or more sheets of electroactive material that exhibits a change in size and/or shape when stimulated by an electric field (e.g., poly-paraphenylene terephthalamide (KEVLAR® or TWARON®), or similar materials). In other embodiments, the exterior portion 122 is formed of a resilient material (e.g., an elastomer such as rubber).

For example, in embodiments, the blades 112 are configured to change length from a compact state (FIG. 5C) to a final state (FIG. 5D) in response to the interior portion 124 being stimulated by an electric field. In some of these embodiments, the change in length also occurs in response to the exterior portion 122 reaching at, about, or above the transition temperature. The blades 112a, 112b include respective lengths (La), (Lb) defined by a distance between an outermost point proximate the distal end 132 (i.e. distal to the shaft 116) of the blades 112 and an innermost point proximate the proximal end 130 (i.e., proximal to the shaft 116) of the blades 112. In some embodiments, the length (La) of the blade 112a is the same as the length (Lb) of the blade 112b in both the compact state and the final state. In additional embodiments, the length (La) may be different than the length (Lb) in the compact state, in the final state, or in both the compact and final states. As non-limiting examples, the length of the blades 112a, 112b may be from about 5 mm to about 25 mm, such as from about 7 mm to about 20 mm, and more particularly from about 9 mm to about 15 mm (e.g., about 11 mm). In some embodiments, the blades 112a, 112b include an overall length of about 9 mm in the compact state and expand to about 13 mm in the final state. In other embodiments, the blades 112a, 112b include an overall length of about 10.5 mm in the compact state and expand to about 18 mm in the final state. In other embodiments, the blades 112a, 112b include an overall length from 9 mm to 10.5 mm in the compact state and expand to an overall length from 13 mm to 18 mm in the final state.

Referring now to FIG. 5C, in some embodiments, the exterior portion 122 of the blades 112 includes corrugated regions 134 positioned about at least a portion of a perimeter of the blade 112 defined by the exterior portion 122. The corrugated regions 134 include a spring-like (e.g., helical) or pleated configuration. In some embodiments, the entire perimeter of the blade 112 defined by the exterior portion 122 of the blades 112 includes a corrugated structure. In a compact state, as shown in FIG. 5C, the corrugations or coils of the corrugated regions 134 of the blades 112 are relatively closely spaced. In some embodiments, the blades 112 are spaced apart from 3 mm to 5 mm apart. By spacing the blades 112 relatively close together, turbulence of blood flow may be minimized.

Referring now to FIG. 5D, in some embodiments, in response to applied energy (e.g., an applied electrical field), the electroactive material within the interior portion 124 of the blades 112 expands in length transitioning the blades 112 from the compact state to the final state. The corrugated regions 134 of the exterior portion 122 are configured to accommodate the change in shape of the electroactive material by flattening and expanding the corrugations, such as into a smooth perimeter, as shown in FIG. 5D, resulting in an increase in the length (La), (Lb) of the blades 112a, 112b. In some embodiments, a width of the blades 112a, 112b is also expanded in response to the corrugations flattening and expanding.

In some embodiments, in response to applied energy (e.g., heat within the subject's body) resulting in the shape memory material of the corrugated regions 134 of the exterior portion 122 (e.g., blade frame) reaching at, about, or above the transition temperature, the corrugated regions 134 flatten and expand from a corrugated arrangement to a non-corrugated/smooth arrangement, as shown in FIG. 5D, causing the length resulting in an increase in the length (La), (Lb) of the blades 112a, 112b

In some embodiments, a computational fluid dynamics simulation device may be utilized to analyze all available patient and device data and determine the ideal impeller shape, length, speed, angle of deflection, curvature, power usage, and more for the situation and goals at hand. The final/remembered shape of the blades 112 is then set, by any of the methods disclosed herein, based on the analysis performed by the computational fluid dynamics simulation device. For example, the impeller blade length, shape, coil amount, curvature, and/or overall form may be selectively changed as needed utilizing an electrical field to result in bending deformation to a pre-determined different shape and size that is most ideal for a given situation.

In some embodiments, the impeller blade length, shape, coil amount, curvature, and/or overall form is selectively changed utilizing a light activated shape change material to result in bending deformation to a pre-determined different shape and size in response to an applied light, and can be selectively returned to the original shape also to facilitate removal percutaneously if desired.

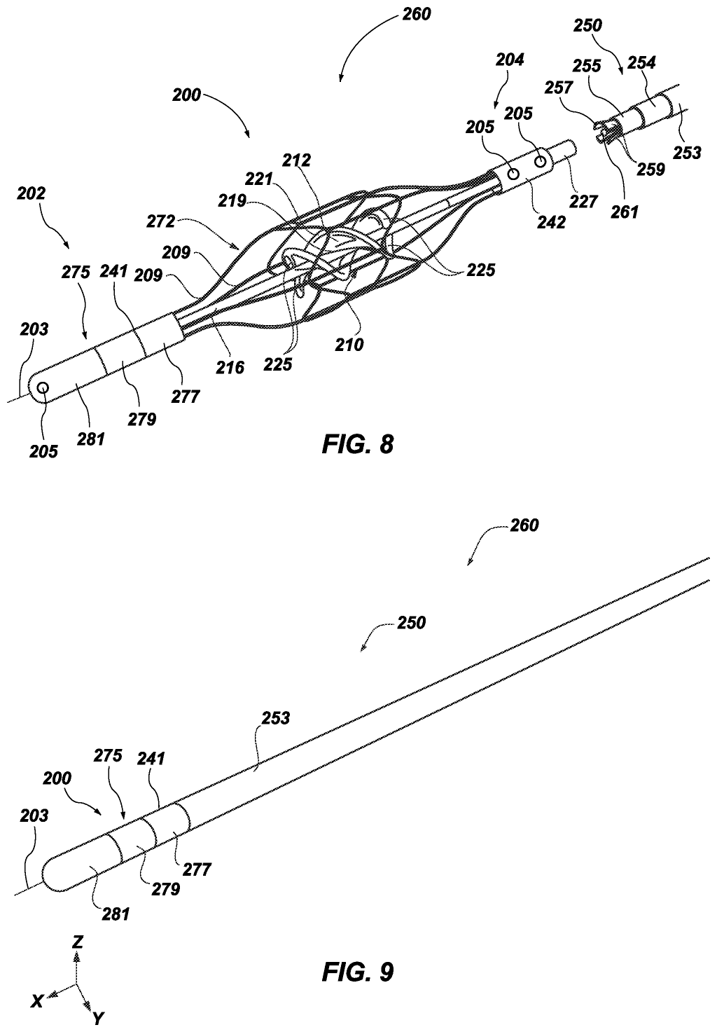

FIG. 8 illustrates an embodiment of the circulatory assist device 260 including a wireless circulatory assist pump 200 in an expanded state, according to embodiments of this disclosure. Referring to FIG. 8, in some embodiments, the circulatory assist device 260 includes a wireless circulatory assist pump 200 and a placement catheter 250.

In some embodiments, the wireless circulatory assist pump 200 generally includes a distal end 202, a proximal end 204 (e.g., docking end), an impeller 210, a stent cage 272, and a motor system 275. In embodiments, the motor system includes a battery 277, circuitry 279, and a motor 281 configured to rotate the impeller 210. In some embodiments, the circuitry 279 includes a wireless charging circuit, a communications circuit, and a control circuit. As shown in FIG. 24, the battery 277, the circuitry 279, and the motor 281, may all be located at or adjacent to an end of the wireless circulatory assist pump 200, such as the distal end 202 or the proximal end 204, but it will be understood that one or more, or all, of the battery 277, the wireless charging circuit, the communications circuit, the control circuit, and the motor 281 may alternatively be located at distinct locations along a length of the wireless circulatory assist pump 200. With the wireless charging circuit, the communications circuit, the control circuit, the motor 281, and the battery 277, the wireless circulatory assist pump 200 can operate within an artery of a subject/patient without a wired connection, such as via a catheter, to components outside of the subject/patient. In some embodiments, the circulatory assist pump 200 includes at least one casing, such as a distal casing 241 at the distal end 202 and a proximal casing 242 at the proximal end 204. In some embodiments, the motor system 275 is positioned within the at least one casing 241, 242.

In some embodiments, the wireless circulatory assist pump 200 also includes a gripping portion 227 positioned at the proximal end 204. In some embodiments, the gripping portion 227 is unitarily formed with the proximal casing 242. In other embodiments, the gripping portion 227 is joined to the proximal casing 242. The gripping portion 227 is configured to be coupled to the placement catheter 250.

In some embodiments, the wireless circulatory assist pump 200 includes a single impeller 210 including a blade 212 and a shaft 216. In some embodiments, the blade 212 and the shaft 216 are formed as a single unitary structure. Accordingly, the impeller 210 may be substantially free of any fasteners or separate mechanical mechanisms (e.g., cam mechanisms, springs, etc.). In some of these embodiments, a portion of the shaft 216 forms an inner radial structure of the blade 212. In other embodiments, the wireless circulatory assist pump 200 includes multiple (e.g., two or more) impellers 210 arranged in series. In further embodiments, the impeller 210 includes multiple blades 212 offset axially and unitarily formed with the shaft 216. Accordingly, one revolution of the shaft 216 corresponds to one revolution of the blade(s) 212. The impeller 210, including the blade 212 and the shaft 216, is configured to rotate about a central longitudinal axis 203 (e.g., in a circumferential direction) thereof.

In some embodiments, the wireless circulatory assist pump 200 includes micro coils 206. In some embodiments, the micro coils 206 are configured to control aortic tissue protein expressions and to increase smooth muscle mass and to control pulsations of natural aortic muscle, a cellular muscle-based “second heart.” For example, pacing the timed electrical pulse signals may be utilized to trigger contractions of smooth muscle so to make the natural aorta a beating “second heart” optimized with native pulsatile flow. In some embodiments, the micro coils 206 are configured to control chronic inflammation and blood pressure with real time reads and adjustments. In some embodiments, the micro coils 206 are configured to transmit bioelectric signals to control and/or modify regenerative protein expressions, such as to increase elasticity, control blood pressure, improve organ health, and control inflammation.

FIG. 9 illustrates the circulatory assist device 260 of FIG. 8 with the wireless circulatory assist pump 200 in a collapsed state (closed configuration). Referring to FIGS. 8 and 9, the circulatory assist pump 200 is configured to transition between an expanded state (refer to FIG. 8) and a collapsed state (refer to FIG. 9). In some embodiments, the impeller 210 is configured to collapse from the expanded state and be stored within the at least one casing 241, 242. The impeller 210, and in particular, the blade 212, is configured to collapse radially from a deployed state (e.g., expanded or open state), as shown in FIG. 8, to a stowed state (e.g., collapsed or closed state) to a radial dimension that is less than an inner diameter of the at least one casing 241, 242 to be contained therein, as shown in FIG. 9, and vice versa. In embodiments, the shaft 216 is configured to collapse in the axial direction thereof to transition between the expanded state and the collapsed state.

Referring again to FIG. 8, in embodiments, the blade 212 includes helical airfoil with a generally helical shape (e.g., a shape of an auger) that is configured to regulate blood flow, such as increase blood flow in the artery of the subject/patient in an axial direction relative to the longitudinal axis 203. The helical airfoil includes a fixed edge 219 (e.g., fixed edge) joined to the shaft 216, a free edge 221 (e.g., radially outermost edge) defining a helical frame, and an interior body connected to and interposed between the free edge 221 and the fixed edge 219. In some embodiments, the free edge 221 is reinforced relative to the fixed edge 219, such as being thicker and/or of a stronger material. In some embodiments, the blade 212 includes reinforced ends 225 at distal and proximal axial ends thereof, such as being thicker and/or of a stronger material. The reinforced ends 225 and the reinforced free edge 221 may improve the resilience of the blade 212, and may help the blade retain the helical shape while facilitating blood circulation within an artery. In operation, the blade 212 (e.g., the helical airfoil) may facilitate about 4.5 liters per minute of blood flow (estimated) at a rotational speed of about 4,500 RPM. In some embodiments, the blade 212 is controlled to facilitate a range of flow from 0.5 liters per minute to 5.5 liters per minute while operating below 9,000 RPM. In some of these embodiments, the blade 212 is controlled to facilitate this range of flow while operating below 6,500 RPM.

In some embodiments, the impeller 210 including the blade 212 is formed of a material with a sufficient spring radial force within the material to maintain the shape thereof, such as the helical shape, while facilitating blood circulation within the artery, and the material of the impeller 210 is sufficiently flexible to fold and/or compress when the at least one casing 241, 242 is moved axially over the impeller 210. In other embodiments, the impeller 210 is formed of a shape changing material and is configured to change shapes. For example, in some of these embodiments, the impeller 210 is configured to selectively transition between the deployed state, the undeployed state, and a partially deployed state. In some of these embodiments, the impeller 210, and in particular, the blade 212 is configured to twist/coil to selectively vary the pitch thereof. For example, the blade 212 is configured to expand or contract away from/towards the central longitudinal axis 203 of the impeller 210 while also changing an angle between the blade 212 and the shaft 216. In some of these embodiments, the blade 212 is also configured to bend and alter a curvature of the blade 212.

The stent cage 272 may be the same or similar to the stent cage 172 described above. The stent cage 272 is configured to expand radially to contact in inner wall of the artery and securely position the wireless circulatory assist pump 200 within the artery, while maintaining the pulsatility of the artery. In some embodiments, the stent cage 272 is also configured to expand axially. Further, the stent cage 272 is configured to collapse radially inward and axially to be stowed within the at least one casing 241, 242 with the impeller 210.

The stent cage 272 may be of a size and shape to allow a highly open blood flow when the stent cage 272 is in an expanded state within the subject's artery/blood vessel(s) (e.g., aorta). Furthermore, the stent cage 272 is configured to exhibit a balance of flexibility and rigidity. In particular, the stent cage 272 is sufficiently rigid to provide a radial force against an inner wall of the subject's artery while the stent cage 272 is in the expanded state to positionally secure the wireless circulatory assist pump 200, and the stent cage 272 is sufficiently flexible/deformable to flex with a natural pulsatility of the subject's artery.

In some embodiments, the stent cage 272 includes wire elements 209 that extend between the distal end 202 and the proximal end 204 and expand radially from each end to form a cage for the blade 212. The cage structure of the wire elements 209 is configured to provide space within the artery for the blade 212 to turn and to prevent contact between the blade 212 and the inner wall of the artery. In some embodiments, the cage includes axially extending wire elements 209 that bend out radially, which are connected to wire elements 209 are generally arranged to extend circumferentially in a sinusoidal wave pattern.

The wire elements 209 include a circular, oval, or polygonal (such as a square) cross-section. Additionally, in some embodiments, the wire elements 209 include a size (e.g., diameter, length, width) within a range of from about 0.1 millimeters (mm) to about 1 mm, such as within a range of from about 0.2 mm to about 0.7 mm, within a range of from about 0.3 mm to about 0.6 mm, (e.g., about 0.5 mm).

In some embodiments, the stent cage 272 is formed of a material with a sufficient spring radial force within the material to maintain the shape thereof while expanded radially outward against an inner wall of the artery, and the material of the stent cage 272 is sufficiently flexible to fold and/or compress when the at least one casing 241, 242 is moved axially over the stent cage 272. In other embodiments, the stent cage 272 is formed of a shape memory material with a “remembered” shape being the stent cage 208 in the expanded state. Accordingly, in these embodiments, the stent cage 272 is biased radially outward after being inserted into the subject's artery, and being removed from the at least one casing 241, 242. In the latter embodiments, the stent cage 208 expands radially outward after being at or above the transition temperature (e.g., about 37° C.). In some embodiments, while the stent cage 208 is in the expanded state within the subject's artery, the radial force provided by the wire elements 209 of the stent cage 272 against the inner wall of the subject's aorta is within a range of from about 0.1 Newton (N) to about 1 N, such as within a range of from about 0.2 N to about 0.8 N, within a range of from about 0.3 N to about 0.7 N, within a range of from about 0.4 N to about 0.6 N (e.g., about 0.5 N). In some embodiments, radial force applied by the wire elements 209 of the stent cage 272 to the inner wall of the subject's artery is sufficient to embed the wire elements 209 at least partially within the inner wall of the subject's artery, and in some embodiments, an entire thickness of the wire elements 209 is embed into the inner wall of the subject's artery resulting in the opening defined within the embedded wire elements 209 of the stent cage 272 is substantially the same size (e.g., area) as the subject's artery. The wire elements 209 of the stent cage 272 being flush within inner wall of the artery may lessen the risks associated with turbulence and obstruction to blood flow.

In some embodiments, the stent cage 272, in the expanded state, is configured to distend the subject's artery by an amount within a range from up to 2 mm (e.g., about 0.5 mm, about 1.0 mm, about 1.5 mm, or about 2 mm) to further secure the position of the stent cage 172 within the subject's artery.

As previously discussed, the impeller 210 and the stent cage 272 are configured to expand radially outward from the central longitudinal axis 203 and collapse radially inward toward the central longitudinal axis 403. Accordingly, in response to the wireless circulatory assist pump 200 being withdrawn into a placement catheter (e.g., placement catheter 250), the axial movement of the placement catheter collapses the stent cage 272 cage and the impeller 210 into the stowed position.

In some embodiments, the wireless circulatory assist pump 200 includes one or more sensors 205. The one or more sensors 205 may include flow sensors (to measure blood flow), such as hemodynamic sensors, temperature sensors, pressure sensors, flow meters, rotational speed sensors (for measuring the RPMs of the impeller 210), and the like.

In some embodiments, the motor 281 is a miniature brushless direct current (DC) motor. For example, the motor 281 may be a miniature brushless DC motor such as available under the tradename “EC6” from Maxon Precision Motors, Inc. of Foster City, Calif. USA.

In some embodiments, the battery 277 is a rechargeable battery, such as a lithium-ion battery. For example, the battery 277 may be a 3 milliamp hour (mAh) lithium-ion battery available under the tradename “CONTIGO” from EaglePicher Technologies of Joplin, Mo. USA. For another example, the battery 277 may be a 3 mAh lithium-ion battery available under the tradename “MICRO3-QL0003B” from Quallion LLC of Sylmar, Calif. USA. It will be understood, however, that the battery 277 may be of any suitable chemistry and/or type, including non-chemical electric power storage devices, such as a capacitor (e.g., a supercapacitor, ultracapacitor, or double-layer capacitor).

The wireless charging circuit is configured to produce an electric current in response to an applied electric field, magnetic field, and/or electromagnetic field. The wireless charging circuit is electrically connected to the battery 277 and is configured to charge the battery 277. For example, in some embodiments, the wireless charging circuit includes an induction coil and is configured for energy to be transferred thereto via inductive coupling. For another example, in some embodiments, the wireless charging circuit includes one or more antennas and energy is transferred thereto via electromagnetic waves (e.g., radio waves).

The communication circuit is configured to send and receive data via wireless communication. For example, the communication circuit may be configured to send and receive data utilizing radio communication (e.g., WiFi, Bluetooth, etc.). In some embodiments, the communication circuit may be utilized to send data collected from one or more sensors 205 of the wireless circulatory assist pump 200. For example, the communication circuit may be utilized to send data relating to the rotational speed of the pump, upstream and downstream fluid pressures, battery charge status, motor status, impeller status, and/or other measured conditions.

The control circuit may be utilized to control certain operations of the wireless circulatory assist pump 200. In some embodiments, the control circuit may be utilized to control the rotational speed of the motor 281, the shape of the impeller 210, the deployment of the blades 212, the stowing of the blades 212, the angle of the blades 212, and/or other operations of the circulatory assist pump 200.

In some embodiments, the circuitry 179 includes one or more application-specific integrated circuit (“ASIC”) chips. For example, one or more of the charging circuit, the communication circuit, and the control circuit may be provided as one or more ASIC chips.

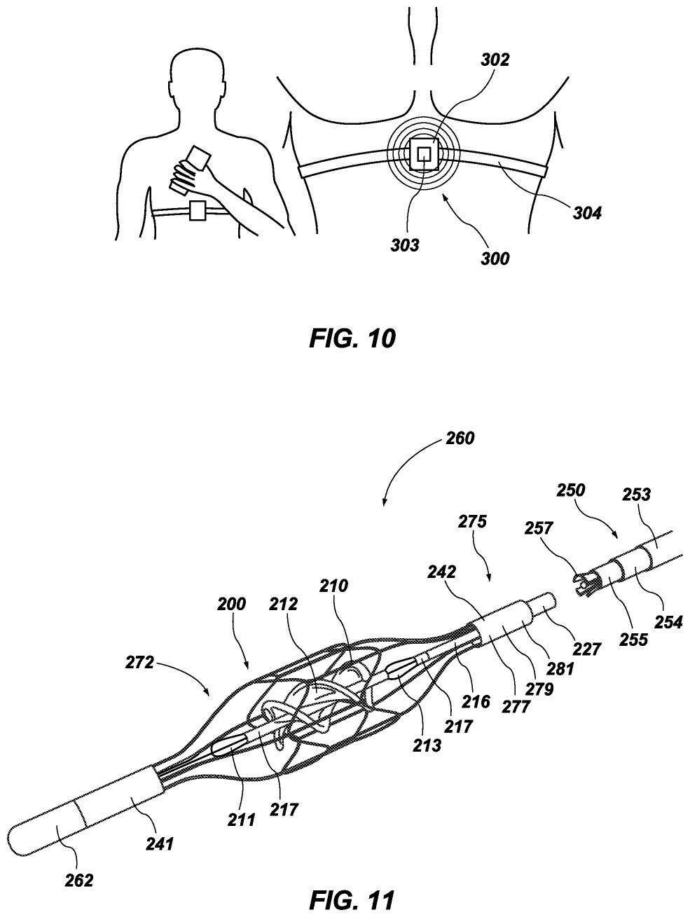

FIG. 10 illustrates an embodiment of a control system 300. In some embodiments, the control system 300 is part of the circulatory assist device 260. In some embodiments, the control system 300 includes a controller 302 and a strap 304 configured to position the controller 302 on a body of a subject. The controller 302 includes circuitry 303. In some embodiments, the circuitry 303 is configured to communicate wirelessly with the communication circuit of the wireless circulatory assist pump 200 (e.g., receive sensor data from and send instructions to the wireless circulatory assist pump 200). In some embodiments, the circuitry is also configured to apply an electric field, a magnetic field and/or an electromagnetic field to that is used by the wireless charging circuit to charge the battery 277 of wireless circulatory assist pump 200). In some embodiments, the circuitry 303 includes an induction coil and is configured to transfer energy via inductive coupling to an induction coil of the wireless circulatory assist pump 200 without coil to coil alignment of the induction coils. For another example, in some embodiments, the circuitry includes one or more antennas and energy is transferred therefrom via electromagnetic waves (e.g., radio waves).

In some embodiments, the circuitry 303 is configured to control the various aspects of the wireless circulatory assist pump 200 disclosed herein. In some of these embodiments, the instructions sent to the wireless circulatory assist pump 200 include signals to control a shape of the impeller 210, a length of the blades 212, a diameter of the stent, rotational speed of the impeller 210, an angle of blade deflection of the blades 212, the bioelectric transmissions from the micro coils 206, and any other aspect of the wireless circulatory assist pump 200.

Referring again to FIGS. 8 and 9, placement catheter 250 is configured to connect to the wireless circulatory assist pump 200 to facilitate introduction and removal of the wireless circulatory assist pump 200 into a subject's artery/blood vessel(s) (e.g., aorta). In some embodiments, the placement catheter 250 includes an outer sheath 253, an intermediate sheath 254, an inner sheath 255, and a gripper 257. The outer sheath 253, the intermediate sheath 254, and/or the inner sheath 255 may include an interior lining comprising expanded polytetrafluoroethylene (ePTFE).

The gripper 257 is positioned within the inner sheath 255 and is configured to extend out from the inner sheath and grasp the gripping portion 227 of the wireless circulatory assist pump 200 and maintain the grasp while components of the wireless circulatory assist pump 200, such as the stent cage 272 and the impeller 210 are positioned within the outer sheath 253 of the placement catheter 250. In some embodiments, the gripper 257 includes one or more fingers 259 (four shown) surrounding an inner member 261 (e.g., a pin). In some embodiments, the outer sheath 253 is configured to move axially relative to the intermediate sheath 254, and/or the inner sheath 255. Furthermore, in some embodiments, the gripper 257 is configured to move axially relative to each of the outer sheath 253, the intermediate sheath 254, and the inner sheath 255.

In some embodiments, the fingers 259 are biased radially outward. Thus, in response to being extended circumferentially relative to the inner sheath 255, the intermediate sheath 254, and the outer sheath 253, the tips of the fingers 336 extend radially outward and apart from one another. The fingers 259 are configured to receive the gripping portion 227 of the wireless circulatory assist pump 200 therebetween. In some embodiments, the gripping portion 227 includes a hole formed therein that is configured to receive the inner member 261 when the gripping portion 227 is received within the fingers 259. In some embodiments, the hole in the gripping portion includes a taper that is configured to guide the coupling between the gripping portion 227 and the gripper 257. Accordingly, the proximal end 204 of the wireless circulatory assist pump 200 is coupled to and aligned with the placement catheter 250.

In some embodiments, the outer sheath 253, intermediate sheath 254, and the inner sheath 255 are configured to move axially over the fingers 336, which biases the fingers 259 inward. Thus, in these embodiments, with the gripping portion or end 227 received within the gripper 257, the fingers 259 are biased inward due to the relative movement between the gripper 257 and that of the inner member 255/intermediate sheath 254/outer sheath 253 which causes the fingers 259 to grip the gripping portion 227, such as via an interference condition created by the radially inward biasing of the fingers 259, coupling the wireless circulatory assist pump 200 and the placement catheter 250 together.

In some embodiments, the outer sheath 253 includes an inner diameter that is larger than an outer diameter of the proximal casing 242 and is configured to translate axially relative to the intermediate sheath 254, and the outer sheath 253 is configured to receive the proximal casing 242, the impeller 210, and the stent cage 272 therein. The impeller 210 and the stent cage 272 are configured to collapse and stow within the outer sheath 253. In some embodiments the impeller 210 and the stent cage 272 are formed of shape memory material and exhibit sufficient flexibility to naturally fold and conform within at least one of the distal casing 241, the proximal casing 242, and the outer sheath 332. For example, in one embodiment, the impeller 210 and the stent cage 272 are stowed completely within the outer sheath 253. In another embodiment, the impeller 210 and the stent cage 272 are stowed partially within at least one of the distal casing 241 and the proximal casing 242 and the remainder of the impeller 210 and the stent cage 272 is stowed within the outer sheath 253.

In some embodiments, the outer sheath 253 is configured to abut an edge of the distal casing 241 while the wireless circulatory assist pump 200 is in the collapsed state (shown in FIG. 9) and partially contained within the outer sheath 253.

The wireless circulatory assist pump 200 is configured to transition from the collapsed state (e.g., stowed state) (FIG. 9) to the expanded state (e.g., deployed state) (FIG. 8), and vice versa. To facilitate introduction and removal, the circulatory assist pump 200 is in the collapsed state (FIG. 9). After inserting the wireless circulatory assist pump 200 into the subject (e.g., within the subject's femoral artery) and positioning the wireless circulatory assist pump 200 in a desired location (e.g., above the subject's renal arteries in the descending aorta), the circulatory assist pump 200 transitions from the collapsed state (FIG. 9) to the expanded state (FIG. 8). After the wireless circulatory assist pump 200 is in the expanded state (FIG. 8), the placement catheter 250 is disconnected from the wireless circulatory assist pump 200 and withdrawn from the subject. Furthermore, in some embodiments, the wireless circulatory assist pump 200 is activated (e.g., via wireless energy or battery power) to rotate the blade 212 to facilitate blood circulation within the subject.

FIG. 11 illustrates an embodiment of the circulatory assist device 260 of FIGS. 8 and 9 including at least one vibrating component 211, 213, according to embodiments of this disclosure. In some embodiments, the circulatory assist device 260 illustrated in FIG. 11 includes the same or similar features to those described above with regards to FIGS. 8 and 9. Referring to FIG. 11, in some embodiments, the wireless circulatory assist pump 200 includes at least one vibrating component 211, 213. In the embodiment illustrated in FIG. 11, the wireless circulatory assist pump 200 includes a vibrating component 211 upstream (relative to an intended blood flow direction) of the blade 212, positioned between the blade 212 and a tip 262/distal casing 241 of the wireless circulatory assist pump 200 and positioned within the stent cage 272. In some embodiments, the wireless circulatory assist pump 200 also includes a vibrating component 213 downstream (relative to an intended blood flow direction) of the blade 212, positioned between the blade 212 and the proximal casing 242 of the wireless circulatory assist pump 200 and positioned within the stent cage 272.

In some embodiments, a portion 217 of the shaft 216 is integrated into the impeller 210. In some of these embodiments, the at least one vibrating component 211, 213 is in a position chosen from among extending from (vibrating component 211) the portion 217 of the shaft 216 and integrated within (vibrating component 213) the portion 217 of the shaft 216. In some embodiments, the shaft 216 extends from the motor 281, beyond the blade 212 and terminates at the vibrating component 211 without reaching an opposing casing 241, 242. While the embodiment illustrated in FIG. 11 illustrates the motor within the proximal casing 242 and the shaft 216 terminating prior to the distal casing 241, in other embodiments, the motor 281 is positioned in the distal casing 241 and the shaft 216 terminates prior to reaching the proximal casing 242. In other embodiments, the shaft 216 extends from within the proximal casing 242 to within the distal casing 241, being supported by the motor 281 at one end and a bearing at the other end (such as in the embodiment illustrated in FIG. 8).

The at least one vibrating component 211, 213 is configured to vibrate, and in embodiments, is configured to provide harmonic vibration of the wireless circulatory assist pump 200 and/or components thereof. Inducing vibration of the wireless circulatory assist pump 200 and/or components thereof may reduce the possibility of or prevent thrombosis (i.e., blood clots) from forming within the artery at the wireless circulatory assist pump 200 and/or components thereof.

In some embodiments, the at least one vibrating component 211, 213 includes a piezoelectric generator. For example, in some of these embodiments, the at least one vibrating component 211, 213 includes a vibrational piezoelectric crystal positioned within a tear drop element. In some embodiments, the at least one vibrating component 211, 213 is configured to reach harmonic resonance with the blood clot risk stagnation points of the various components of the wireless circulatory assist pump 200. In some embodiments, the at least one vibrating component 211, 213 is configured to reach the harmonic resonance in conjunction with any vibration caused by the rotation of the impeller 210. In some embodiments, the vibrational output of the at least one vibrating component 211, 213 is modified in response to a rotational speed of the impeller 210. In some of these embodiments, the vibrational output of the at least one vibrating component 211, 213 is also controlled in response to natural vibrations of the body of the subject. In some embodiments, the at least one vibrating component is controlled by the circuitry 279. In some of these embodiments, the circuitry 303 of the control system 300 is configured to send control signals to the circuitry 279 to control the vibrational output of the at least one vibrating component 211, 213. In other embodiments, the circuitry 279 is configured to control the vibrational output of the at least one vibrating component 211, 213 independently.

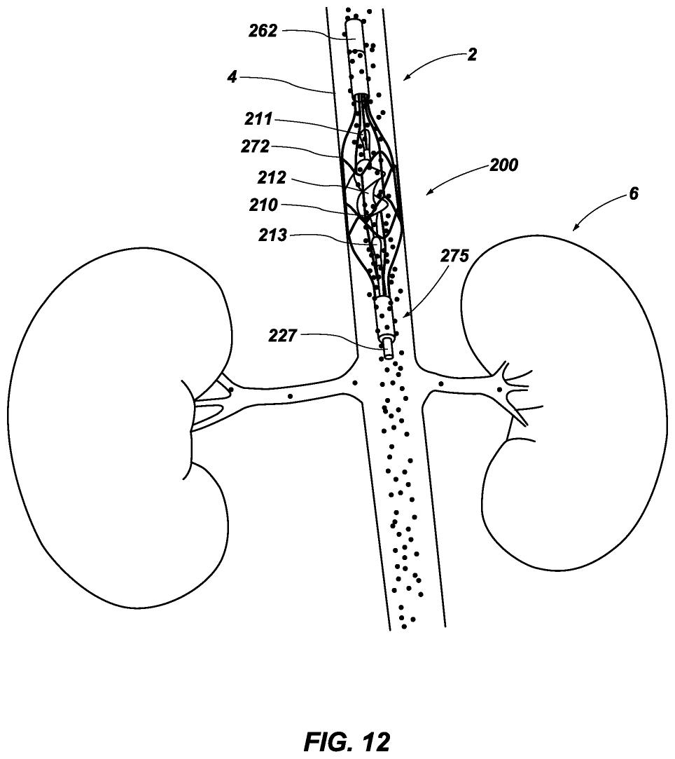

FIG. 12 illustrates the circulatory assist device of FIG. 10 positioned within an artery 2 of a subject. As discussed in detail above, once the wireless circulatory assist pump 200 is inserted into the artery 2 of the subject, the wireless circulatory assist pump 200 is transitioned into an expanded state, the stent cage 272 expands radially outward and contacts a wall 4 of the artery and the blade 212 of the impeller 210 expands to an operational state where the blade 212 thereof is expanded and in a condition for rotating to assist the blood flow within the artery. The blade 212 is rotated to facilitate blood flow through the artery 2. In the embodiment illustrated, the wireless circulatory assist pump 200 is positioned in the artery 2 upstream of the organs (kidneys) 6.

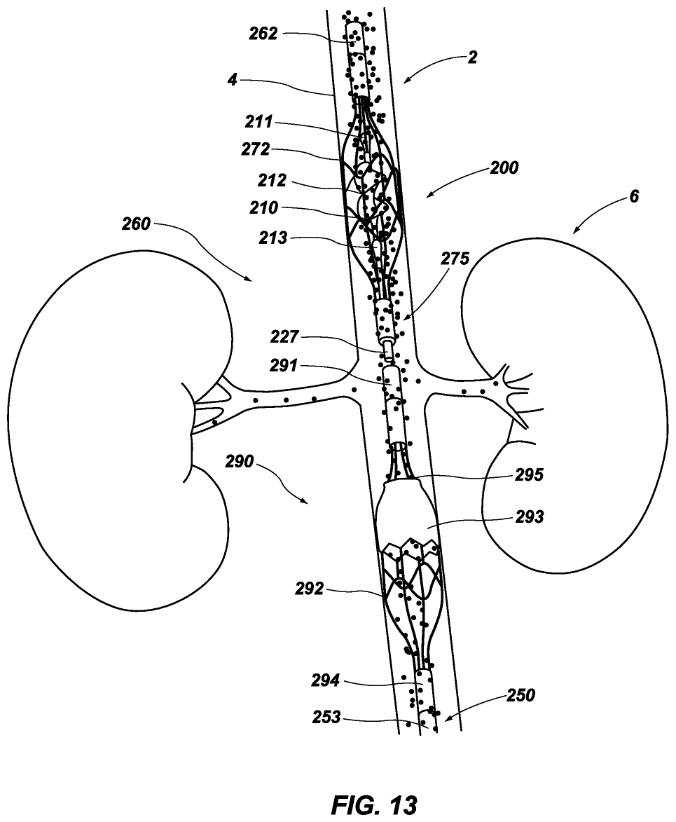

FIG. 13 illustrates another embodiment of a circulatory assist system or device 260 including the wireless circulatory assist pump 200 of FIG. 10 and a lower catheter 290 inserted in the artery 2 of the subject adjacent to the wireless circulatory assist pump 200. Referring to FIG. 13, in some embodiments, the lower catheter 290 includes a lower tip 291, a lower stent cage 292, and a casing 294. The lower stent cage 292 includes a hollow body 293 at an upstream end. In some embodiments, the hollow body 293 is positioned downstream and adjacent to the lower tip 291. The hollow body 293 includes a hollow structure that expands from an upstream end (closer to the lower tip 291) to a downstream end (closer to the casing 294). The hollow structure includes an upstream opening 295 that is configured for blood to flow therethrough, which leads into the hollow structure. The hollow structure also includes a downstream opening 296 that includes a diameter larger than that of the upstream opening 295 and that acts as an exit for blood flow to exit the hollow structure.

In some embodiments, the lower stent cage 292, including the hollow body 293, is formed of a shape memory material, such as any of the shape memory materials disclosed herein including a shape memory alloy and/or a shape memory polymer. Similar to the stent cage 272, in some embodiments, the lower stent cage 292 is configured to expand upon reaching a transition temperature (e.g., about 37° C.), and/or being removed from the outer sheath 253 of the placement catheter 250. In the expanded state, the hollow body 293 is configured to modify the blood flow downstream of the wireless circulatory assist pump 200 to increase blood flow to an organ 6, such as the kidneys, which is downstream of the wireless circulatory assist pump 200 and upstream of the lower catheter 290. Indeed, in some embodiments, the wireless circulatory assist pump 200 is positioned within the artery 2 upstream of the organ 6, while the lower catheter 290 is positioned in the artery 2 downstream of the organ 6.

The lower casing 294 is configured to couple to the placement catheter 250. In some embodiments, the lower casing 294 and the placement catheter 250 include features and similarly couple to that of the gripping portion or end 227 disclosed above.

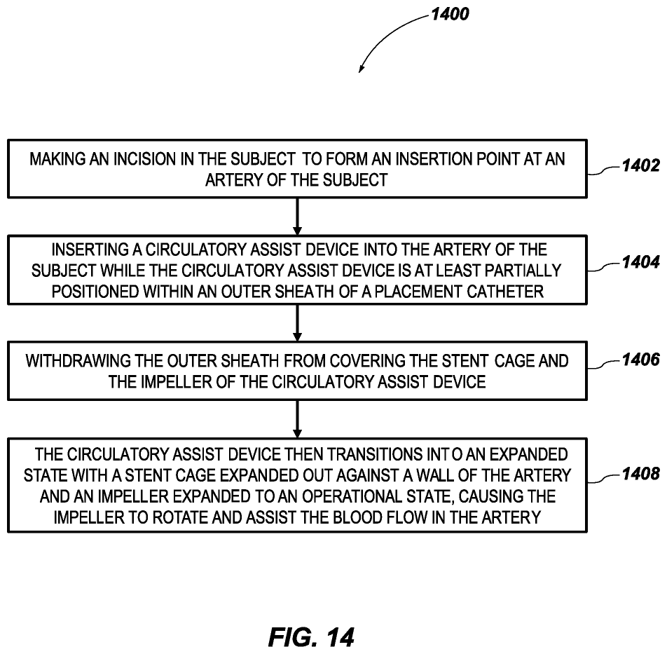

FIG. 14 is a flowchart of a method 1400 for treatment. In embodiments, the treatment includes including the blood flow within a subject. The method includes making an incision in the subject to form an insertion point at an artery of the subject at act 1402. The method also includes inserting a circulatory assist device into the artery of the subject while the circulatory assist device is at least partially positioned within an outer sheath of a placement catheter at act 1404.

The circulatory assist device can be any embodiment of the wireless circulatory assist device 100, 200 disclosed herein. In some embodiments, the circulatory assist device is a wireless circulatory assist device and act 1404 includes gripping the wireless circulatory assist device with a placement catheter and utilizing the placement catheter to position the wireless circulatory assist device within the artery of the subject. In some of these embodiments, act 1404 includes positioning the wireless circulatory assist device upstream of an organ (e.g., kidneys) (FIG. 12) and positioning a lower catheter downstream of the organ (FIG. 13). In some embodiments, positioning the lower catheter downstream of the organ facilitates an increased blood flow into the organ.

The method further includes withdrawing the outer sheath from covering the stent cage and the impeller of the circulatory assist device at act 1406.

The method further includes that the circulatory assist device then transitions into an expanded state with a stent cage expanded out against a wall of the artery and an impeller expanded to an operational state, causing the impeller to rotate and assist the blood flow in the artery at act 1408. In some embodiments, act 1408 includes withdrawing an outer cover of a catheter from over the stent cage and the impeller, which allows the stent cage and the impeller to expand in response to reaching a transition temperature.

In some embodiments, act 1408 includes modifying a speed (RPMs) of the impeller in response to conditions of a body of the subject. These conditions include a measured activity level of the subject (e.g., rest or sleep), current blood flow, temperature, and the like, for the subject.

In some embodiments, the method further includes vibrating at least a portion of the circulatory assist device. As noted above, in some embodiments, the circulatory assist device includes at least one vibrating component that is configured to cause the vibration. By vibrating at least a portion of the circulatory assist device, blood clot formations within the artery at or near the circulatory assist device may be reduced.

In some of the embodiments where the wireless circulatory assist device is positioned upstream of an organ (FIG. 12) and a lower catheter is positioned downstream of the organ (FIG. 13), the lower catheter is removed in response to a fluid being removed from the organ, while the circulatory assist device remains in position within the artery.

In some embodiments, the method yet further includes removing the circulatory assist device from the artery. In some of these embodiments, the wireless circulatory assist device is removed by gripping an end thereof with a placement catheter, positioning the outer cover over the stent cage and the impeller and removing the wireless circulatory assist device while in a collapsed state.

In the Brief Summary, the Detailed Description, the claims below, and in the accompanying drawings, reference is made to particular features (including method acts) of this disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

The description provides specific details, such as components, assembly, and materials in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional components and fabrication techniques employed in the industry. Also note, any drawings accompanying this disclosure are for illustrative purposes only, and are thus not necessarily drawn to scale. Additionally, elements common between figures may retain the same numerical designation.

As used herein, the terms “adapted,” “configured,” and “configuration” refers to a size, shape, material composition, material distribution, orientation, and arrangement of one or more of at least one structure and at least one apparatus facilitating operation of one or more of the structure and the apparatus in a predetermined way.

As used herein, the terms “comprising” and “including,” and grammatical equivalents thereof are inclusive or open-ended terms that do not exclude additional, unrecited elements or method steps, but also include the more restrictive terms “consisting of” and “consisting essentially of” and grammatical equivalents thereof. As used herein, the term “may” with respect to a material, structure, feature, or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

As used herein, the singular forms “a,” “an,” and “the” include the plural forms as well, unless the context clearly indicates otherwise.

As used herein, the term “and/or” includes any and all combinations of one or more of the associated listed items.

As used herein, relational terms, such as “first,” “second,” etc., are used for clarity and convenience in understanding the disclosure and accompanying drawings and does not connote or depend on any specific preference, orientation, or order, except where the context clearly indicates otherwise.

As used herein, the term “about,” when used in reference to a numerical value for a particular parameter, is inclusive of the numerical value and a degree of variance from the numerical value that one of ordinary skill in the art would understand is within acceptable tolerances for the particular parameter. For example, “about,” in reference to a numerical value, may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

The various illustrative logical blocks, modules, and circuits described in connection with the embodiments of the circulatory assist device 100, 200, and in particular, the circuitry 179, 279, 303, disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to embodiments of this disclosure.

The embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternative useful combinations of the elements described, will become apparent to those skilled in the art from the description. Such modifications and embodiments also fall within the scope of the appended claims and equivalents.

### REFERENCES

The contents of each of the following references are incorporated herein by this reference:

U.S. Pat. No. 3,620,212 to Fannon et al.

U.S. Pat. No. 3,786,806 to Johnson et al.

U.S. Pat. No. 3,890,977 to Wilson et al.

U.S. Pat. No. 5,964,771 to Beyar et al.

U.S. Pat. No. 8,277,404 to Einarsson.

U.S. Pat. No. 4,283,233 to Goldstein et al. (Aug. 11, 1981)

U.S. Pat. No. 8,617,239 to Reitan (Dec. 31, 2013),

U.S. Patent Pub. No. 2009/0248141 to Shandas et al. (Published Oct. 1, 2009)

U.S. 2021/0077687 A1 to Leonhardt (Published Mar. 18, 2021),

U.S. 2021/0008263 A1 to Leonhardt

Davor Barić, Croatian Medical Journal, Volume 55(6), December 2014, pages 609-620, DOI: 10.3325/cmj.2014.55.609

1. A circulatory assist device comprising:
a stent cage formed of a first material that is sufficiently rigid to expand radially outward and press against an artery wall of an artery and that is sufficiently deformable to collapse within an outer sheath of a placement catheter; and
an impeller including at least one blade formed of a second material that is sufficiently rigid to expand and retain shape while rotating and assisting blood to flow within the artery and is sufficiently deformable to collapse within the outer sheath with the stent cage.

2. The circulatory assist device of claim 1, wherein the blade includes a helical shape that is maintained by a spring radial force of the second material while in an expanded state outside of the outer sheath.

3. The circulatory assist device of claim 1, wherein the stent cage is sufficiently deformable to flex with a natural pulsatility of the artery of a subject.

4. The circulatory assist device of claim 1, further comprising a casing connected an end of the stent cage, a motor positioned within the casing and configured to rotate the blade, and a shaft connecting the motor to the blade.

5. The circulatory assist device of claim 4, wherein the outer sheath includes an inner diameter that is larger than an outer diameter of the casing and the outer sheath is configured to receive the casing therein with the stent cage and the blade.

6. The circulatory assist device of claim 1, further comprising at least one vibrating component configured to vibrate at least a portion of the circulatory assist device.

7. The circulatory assist device of claim 6, wherein the at least one vibrating component is in a position chosen from among extending from a shaft of the impeller and integrated within the shaft.

8. The circulatory assist device of claim 1, wherein the stent cage and the blade are each formed of a shape memory material and are configured to be in an expanded state in response to being at or above a body temperature of a subject and being removed from the outer sheath.

9. A circulatory assist system comprising:
a placement catheter including an outer sheath; and
a circulatory assist device including
a stent cage formed of a first material that is sufficiently rigid to expand radially outward and press against an artery wall of an artery and that is sufficiently deformable to collapse within the outer sheath, and
an impeller including at least one blade formed of a second material that is sufficiently rigid to expand and retain shape while rotating and assisting blood to flow within the artery and is sufficiently deformable to collapse within the outer sheath with the stent cage.

10. The circulatory assist system of claim 9, wherein the placement catheter is configured to position the circulatory assist device within an artery of a subject and the outer sheath is configured to move axially relative to the circulatory assist device to withdraw from over the stent cage and the impeller allowing the stent cage and the blade to expand and to cover the stent cage and the impeller causing the stent cage and the blade to collapse therewithin.

11. The circulatory assist system of claim 9, wherein the stent cage is sufficiently deformable to flex with a natural pulsatility of the artery of a subject and the blade includes a helical shape that is maintained by a spring radial force of the second material while in an expanded state outside of the outer sheath.

12. The circulatory assist system of claim 9, wherein the circulatory assist device includes a casing connected to an end of the stent cage, a motor positioned within the casing and configured to rotate the blade, and a shaft connecting the motor to the blade.

13. The circulatory assist system of claim 12, wherein the outer sheath includes an inner diameter that is larger than an outer diameter of the casing and the outer sheath is configured to receive the casing therein with the stent cage and the blade.

14. The circulatory assist system of claim 13, wherein the placement catheter includes a gripper configured to grasp an end of the circulatory assist device while the outer sheath is withdrawn therefrom and is configured to maintain the grasp while the stent cage and the impeller are within the outer sheath.

15. The circulatory assist system of claim 14, further comprising a lower catheter configured to be positioned within an artery of a subject adjacent to and downstream of the circulatory assist device, the lower catheter including a lower stent cage including a hollow body, the hollow body includes a hollow structure that expands from an upstream end to a downstream end and is configured for blood to flow therethrough, the lower catheter including a third shape memory material, the lower catheter being configured to expand in response to reaching a third predetermined temperature and configured to collapse within the outer sheath of the placement catheter.

16. The circulatory assist system of claim 9, wherein the circulatory assist device includes at least one vibrating component configured to vibrate at least a portion of the circulatory assist device.

17. The circulatory assist system of claim 16, wherein the at least one vibrating component is in a position chosen from among extending from the from a shaft of the impeller and integrated within the shaft.

18. A method of treating a subject in need thereof, the method comprising:
making an incision in the subject to form an insertion point at an artery of the subject;
inserting a circulatory assist device into the artery of the subject while the circulatory assist device is at least partially positioned within an outer sheath of a placement catheter, the circulatory assist device including a stent cage formed of a first material that is sufficiently rigid to expand radially outward and press against an artery wall of an artery and that is sufficiently deformable to collapse within the outer sheath, and an impeller including at least one blade formed of a second material that is sufficiently rigid to expand and retain shape while rotating and assisting blood to flow within the artery and is sufficiently deformable to collapse within the outer sheath with the stent cage;
withdrawing the outer sheath from covering the stent cage and the impeller of the circulatory assist device; and
after the circulatory assist device transitions into an expanded state with the stent cage expanded out against the artery wall and the blade expanding to an operational state, causing the impeller to rotate and assist the blood flow in the artery.

19. The method of claim 18, further comprising modifying a speed of the impeller in response to conditions of a body of the subject.

20. The method of claim 18, wherein the circulatory assist device includes at least one vibrating component, the method further comprising vibrating at least a portion of the circulatory assist device with the at least one vibrating component.
